(19) United States
(12) Patent Application Publication
(10) Pub. No.: US 2022/0361760 A1
(43) Pub. Date: Nov. 17, 2022

(54) WEARABLE BAND FOR BIOMARKER TRACKING

(71) Applicant: Jabil Inc., St. Petersburg, FL (US)

(72) Inventors: Yusuf Abu Tayeb Bhagat, St. Petersburg, FL (US); Patrick John Verdon, St. Petersburg, FL (US); Michael Nicholas Arfaras, St. Petersburg, FL (US); Krishnaveni Das, St. Petersburg, FL (US); Mark Edward Sussman, St. Petersburg, FL (US); Stephen Costa, St. Petersburg, FL (US); Jackson Flynn, St. Petersburg, FL (US); Senaka Balasuriya, St. Petersburg, FL (US); Sai Guruva Reddy Avuthu, St. Petersburg, FL (US); Adrian Gutierrez, St. Petersburg, FL (US)

(73) Assignee: Jabil Inc., St. Petersburg, FL (US)

(21) Appl. No.: 17/765,607

(22) PCT Filed: Oct. 2, 2020
(86) PCT No.: PCT/US2020/053953

§ 371 Date: Mar. 31, 2022

Related U.S. Application Data
(60) Provisional application No. 62/909,468, filed on Oct. 2, 2019.

(51) Int. Cl. A61B 5/0205; A61B 5/024; A61B 5/332; A61B 5/00; A61B 5/11

(52) U.S. Cl. CPC A61B 5/0205; A61B 5/02416; A61B 5/02438; A61B 5/332; A61B 5/681; A61B 5/11; A61B 5/743; A61B 2562/0219; A61B 2562/0215

(57) ABSTRACT

Disclosed herein are wearable bands for biomarker tracking and methods for making the wearable bands. The biomarker tracking wearable band having a printed circuit board assembly (PCBA), the PCBA including an electrocardiography (ECG) sensor utilizing printed Silver-Silver Chloride (Ag-AgCl) electrodes and an optical photoplethysmography (PPG) sensor utilizing more than two light emitting diodes (LEDs), and a directly over molded band encasing the PCBA.

### TECHNICAL FIELD

This disclosure relates to electronics and in particular, a wearable band for biomarker tracking, and the like.

### BACKGROUND

Biomarkers are physiological signals and/or measurements and the like which may be used as an indicator of a particular disease state or some other physiological state of an organism. Biomarker tracking devices are capable of measuring multiple physiologic parameters of a patient. These physiologic parameters may include heart rate, electrocardiogram signals, blood volume changes, oxygen saturation, and other like signals and information. The biomarker tracking devices come in a variety of forms including smart watches, mobile phones, wearable devices, and the like. The use of such devices has become ubiquitous as users become more health conscious. The devices may be used in a variety of settings including medical facilities, home, and work, and while walking, exercising and performing other activities. The devices may be costly, need maintenance, and may be difficult to use or interpret. Consequently, there is a need for an easy to use vital signs monitoring device which may be more suitable and adaptable for a variety of environments.

### SUMMARY

Disclosed herein are implementations of wearable bands for biomarker tracking and methods for making the wearable bands.

In implementations, the biomarker tracking wearable band has a printed circuit board assembly (PCBA), the PCBA including an electrocardiography (ECG) sensor utilizing printed Silver-Silver Chloride (Ag-AgCl) electrodes and an optical photoplethysmography (PPG) sensor utilizing more than two light emitting diodes (LEDs), and a directly over molded band encasing the PCB. In implementations, the printed Silver-Silver Chloride (Ag-AgCl) electrodes further comprising a side Ag-AgCl electrode configured to be contacted by a finger, and a bottom Ag-AgCl electrode configured for contact with an appendage, wherein placement of the finger on the side Ag-AgCl electrode completes a circuit for enabling a single-lead ECG readout. In implementations, the biomarker tracking wearable band further comprising an accelerometer configured to monitor user activity including at least step counts and body posture; and a lightpipe configured over the more than two LEDs. In implementations, the more than two light LEDs and one or more photodiodes are on a same plane to implement reflectance oximetry and the one or more photodiodes measure backscatter of light. In implementations, the directly over molded band comprises low temperature silicones mixed with additives including at least a catalyst, a control, an accelerant, and color using a mold machine, wherein the silicone begins to cure when the catalyst in the silicone and the control mix together. In implementations, the PCBA comprises a plurality of holes for alignment in the mold machine.

In implementations, a system for tracking biomarkers including a biomarker tracking wearable band including a printed circuit board assembly (PCBA), the PCBA including an electrocardiography (ECG) sensor utilizing printed Silver-Silver Chloride (Ag-AgCl) electrodes; and an optical photoplethysmography (PPG) sensor utilizing more than two light emitting diodes (LEDs); and a directly over molded band encasing the PCBA; and a device configured to receive data from the biomarker tracking wearable band, the device configured to present heart rate (HR) and heart rate variability (HRV) from the ECG sensor, present HR, HRV and SpO₂ measurements from the PPG sensor; and present blood pressure measurements on the basis of a pulse transit time (PTT) derived from ECG sensor waveform data decomposition and PPG sensor waveform data decomposition. In implementations, the printed Silver-Silver Chloride (Ag-AgCl) electrodes further comprising a side Ag-AgCl electrode configured to be contacted by a finger; and a bottom Ag-AgCl electrode configured for contact with an appendage, wherein placement of the finger on the side Ag-AgCl electrode completes a circuit for enabling a single-lead ECG readout. In implementations, the biomarker tracking wearable band further comprising an accelerometer configured to monitor user activity including at least step counts and body posture and a lightpipe configured over the more than two LEDs, the device configured to present step counts and body posture from the accelerometer. In implementations, the more than two light LEDs and one or more photodiodes are on a same plane to implement reflectance oximetry and the one or more photodiodes measure backscatter of light. In implementations, the directly over molded band comprises low temperature silicones mixed with additives including at least a catalyst, a control, an accelerant, and color using a mold machine, wherein the silicone begins to cure when the catalyst in the silicone and the control mix together. In implementations, the PCBA comprises a plurality of holes for alignment in the mold machine. In implementations, the system further comprising a cloud system configured to receive and transmit data with the device, wherein the data includes at least historical sensor data and biomarker data.

In implementations, method for tracking biomarkers using the biomarker tracking wearable band and the system for tracking biomarkers as described herein.

### BRIEF DESCRIPTION OF THE DRAWINGS

The disclosure is best understood from the following detailed description when read in conjunction with the accompanying drawings and are incorporated into and thus constitute a part of this specification. It is emphasized that, according to common practice, the various features of the drawings are not to-scale. On the contrary, the dimensions of the various features are arbitrarily expanded or reduced for clarity.

FIG. 1 is an example diagram of an architecture for a biomarker tracking wearable band in accordance with certain implementations.

FIG. 1A is an example diagram of a cloud architecture for a biomarker tracking wearable band in accordance with certain implementations.

FIG. 1B is an example flowchart for cloud storage in a cloud architecture for a biomarker tracking wearable band in accordance with certain implementations.

FIG. 1C is an example flowchart for querying cloud storage in a cloud architecture for a biomarker tracking wearable band in accordance with certain implementations.

FIG. 2 is an example diagram of a biomarker tracking wearable band in accordance with certain implementations.

FIG. 3 is an example diagram of a biomarker tracking wearable band in accordance with certain implementations.

FIGS. 4A-B are example diagrams of a biomarker tracking wearable band in accordance with certain implementations.

FIG. 5 is an example diagram of a hardware architecture of a biomarker tracking wearable band in accordance with certain implementations.

FIG. 6 is an example diagram of a software architecture of a biomarker tracking wearable band in accordance with certain implementations.

FIG. 7 with FIGS. 7A, 7B, 7C, 7D, 7E, 7F, and 7G are example diagrams or layouts of a printed circuit board assembly (PCBA) of a biomarker tracking wearable band in accordance with certain implementations.

FIG. 8 is an example diagram or layout of a top view of a PCBA of a biomarker tracking wearable band in accordance with certain implementations.

FIG. 9 is an example diagram or layout of a bottom view of a PCBA of a biomarker tracking wearable band in accordance with certain implementations.

FIG. 10 is an example diagram or layout of a top view of a PCBA without the battery of a biomarker tracking wearable band in accordance with certain implementations.

FIG. 11 is an example diagram or layout of a side view of a PCBA of a biomarker tracking wearable band in accordance with certain implementations.

FIG. 12 is an example photograph of a perspective view of a PCBA in a holder of a biomarker tracking wearable band in accordance with certain implementations.

FIG. 13 is an example photograph of an over molded biomarker tracking wearable band in accordance with certain implementations.

FIGS. 14A and 14B are photographs of a mold for use in over molding for a biomarker tracking wearable band in accordance with certain implementations.

FIGS. 15A-F are example diagrams of interface screens on a device for interacting with a biomarker tracking wearable band in accordance with certain implementations.

### DETAILED DESCRIPTION

The figures and descriptions provided herein may be simplified to illustrate aspects of the described embodiments that are relevant for a clear understanding of the herein disclosed processes, machines, manufactures, and/or compositions of matter, while eliminating for the purpose of clarity other aspects that may be found in typical similar devices, systems, compositions and methods. Those of ordinary skill may thus recognize that other elements and/or steps may be desirable or necessary to implement the devices, systems, compositions and methods described herein. However, because such elements and steps are well known in the art, and because they do not facilitate a better understanding of the disclosed embodiments, a discussion of such elements and steps may not be provided herein. However, the present disclosure is deemed to inherently include all such elements, variations, and modifications to the described aspects that would be known to those of ordinary skill in the pertinent art in light of the discussion herein.

Embodiments are provided throughout so that this disclosure is sufficiently thorough and fully conveys the scope of the disclosed embodiments to those who are skilled in the art. Numerous specific details are set forth, such as examples of specific aspects, devices, and methods, to provide a thorough understanding of embodiments of the present disclosure. Nevertheless, it will be apparent to those skilled in the art that certain specific disclosed details need not be employed, and that embodiments may be embodied in different forms. As such, the exemplary embodiments set forth should not be construed to limit the scope of the disclosure.

The terminology used herein is for the purpose of describing particular embodiments only and is not intended to be limiting. For example, as used herein, the singular forms “a”, “an” and “the” may be intended to include the plural forms as well, unless the context clearly indicates otherwise. The terms “comprises,” “comprising,” “including,” and “having,” are inclusive and therefore specify the presence of stated features, steps, operations, elements, and/or components, but do not preclude the presence or addition of one or more other features, steps, operations, elements, components, and/or groups thereof.

The steps, processes, and operations described herein are thus not to be construed as necessarily requiring their respective performance in the particular order discussed or illustrated, unless specifically identified as a preferred or required order of performance It is also to be understood that additional or alternative steps may be employed, in place of or in conjunction with the disclosed aspects.

Yet further, although the terms first, second, third, etc. may be used herein to describe various elements, steps or aspects, these elements, steps or aspects should not be limited by these terms. These terms may be only used to distinguish one element or aspect from another. Thus, terms such as “first,” “second,” and other numerical terms when used herein do not imply a sequence or order unless clearly indicated by the context. Thus, a first element, step, component, region, layer or section discussed below could be termed a second element, step, component, region, layer or section without departing from the teachings of the disclosure.

As used herein, the terminology “determine” and “identify,” or any variations thereof includes selecting, ascertaining, computing, looking up, receiving, determining, establishing, obtaining, or otherwise identifying or determining in any manner whatsoever using one or more of the devices and methods are shown and described herein.

As used herein, the terminology “example,” “the embodiment,” “implementation,” “aspect,” “feature,” or “element” indicates serving as an example, instance, or illustration. Unless expressly indicated, any example, embodiment, implementation, aspect, feature, or element is independent of each other example, embodiment, implementation, aspect, feature, or element and may be used in combination with any other example, embodiment, implementation, aspect, feature, or element.

As used herein, the terminology “or” is intended to mean an inclusive “or” rather than an exclusive “or.” That is unless specified otherwise, or clear from context, “X includes A or B” is intended to indicate any of the natural inclusive permutations. That is if X includes A; X includes B; or X includes both A and B, then “X includes A or B” is satisfied under any of the foregoing instances. In addition, the articles “a” and “an” as used in this application and the appended claims should generally be construed to mean “one or more” unless specified otherwise or clear from the context to be directed to a singular form.

As used herein, the terminology “computer” or “computing device” includes any unit, or combination of units, capable of performing any method, or any portion or portions thereof, disclosed herein. For example, the “computer” or “computing device” may include at least one or more processor(s).

As used herein, the terminology “processor” indicates one or more processors, such as one or more special purpose processors, one or more digital signal processors, one or more microprocessors, one or more controllers, one or more microcontrollers, one or more application processors, one or more central processing units (CPU)s, one or more graphics processing units (GPU)s, one or more digital signal processors (DSP)s, one or more application specific integrated circuits (ASIC)s, one or more application specific standard products, one or more field programmable gate arrays, any other type or combination of integrated circuits, one or more state machines, or any combination thereof.

As used herein, the terminology “memory” indicates any computer-usable or computer-readable medium or device that can tangibly contain, store, communicate, or transport any signal or information that may be used by or in connection with any processor. For example, a memory may be one or more read-only memories (ROM), one or more random access memories (RAM), one or more registers, low power double data rate (LPDDR) memories, one or more cache memories, one or more semiconductor memory devices, one or more magnetic media, one or more optical media, one or more magneto-optical media, or any combination thereof.

As used herein, the terminology “instructions” may include directions or expressions for performing any method, or any portion or portions thereof, disclosed herein, and may be realized in hardware, software, or any combination thereof. For example, instructions may be implemented as information, such as a computer program, stored in memory that may be executed by a processor to perform any of the respective methods, algorithms, aspects, or combinations thereof, as described herein. Instructions, or a portion thereof, may be implemented as a special purpose processor, or circuitry, that may include specialized hardware for carrying out any of the methods, algorithms, aspects, or combinations thereof, as described herein. In some implementations, portions of the instructions may be distributed across multiple processors on a single device, on multiple devices, which may communicate directly or across a network such as a local area network, a wide area network, the Internet, or a combination thereof.

As used herein, the term “application” refers generally to a unit of executable software that implements or performs one or more functions, tasks or activities. For example, applications may perform one or more functions including, but not limited to, vital signs monitoring, health monitoring, telephony, web browsers, e-commerce transactions, media players, travel scheduling and management, smart home management, entertainment, and the like. The unit of executable software generally runs in a predetermined environment and/or a processor.

The non-limiting embodiments described herein are with respect to wearable bands and methods for making the wearable bands, where the wearable bands are biomarker tracking wearable bands. The wearable band and method for making the wearable band may be modified for a variety of applications and uses while remaining within the spirit and scope of the claims. The embodiments and variations described herein, and/or shown in the drawings, are presented by way of example only and are not limiting as to the scope and spirit. The descriptions herein may be applicable to all embodiments of the device and the methods for making the devices.

Disclosed herein are implementations of biomarker tracking wearable bands and methods for making the wearable bands. The biomarker tracking wearable band provides multiple sensing modalities including activity monitoring, optical photoplethysmography (PPG) utilizing more than two light emitting diodes (LEDs) and photodiodes or photodetectors, and electrocardiography (ECG) utilizing printed Silver-Silver Chloride (Ag-AgCl) electrodes in a small form factor wearable band. In an implementation, the wearable band can be a wristband, ankle band, and the like. The biomarker tracking wearable band integrates multiple sensing modalities which work in harmony to provide several parameters for wholistic serial monitoring of an individual's health status, for example, ECG, oximetry, and derivative blood pressure measurements on one device.

In an implementation, the biomarker tracking wearable band includes a printed circuit board (PCB) assembly (PCBA) directly over molded in liquid silicone rubber to fabricate a wearable band for monitoring activity from an accelerometer in terms of step counts and body posture (running, walking, stationary), heart rate (HR) and heart rate variability (HRV) from screen printed ECG electrodes for single-lead ECG measurements, HR, HRV and SpO2 measurements from a PPG sensor (reflectance oximetry) comprising three LEDs (red, green and Infrared) and four photodiodes, and blood pressure measurements on the basis of the pulse transit time (PTT) derived from the ECG and PPG waveform data decomposition. In this instance of reflectance oximetry, the LEDs and photodiodes (PDs) are on the same plane and PDs measure backscatter of light.

Data are transferred to mobile or cellular phone applications, which then on the basis of application programming interfaces (APIs) communicate with a cloud backend for long-term data storage and retrieval of archival parameters. In an implementation, the mobile or cellular phone applications provide a visualization of the data.

In an implementation, the biomarker tracking wearable band interfaces with a smartphone application to measure, stream, and record real-time data for providing comprehensive sensing information to user(s). The applications transmit post-processed sensor data to the cloud for storage and retrieval of historical trends to the smartphone application. In an implementation, the mobile or cellular phone applications communicate with the back-end cloud server to store and retrieve historical sensor data for guiding health and wellness decisions of the users.

In an implementation, blood pressure is measured by simultaneous ECG and PPG measurements to derive a pulse transit time (PTT). The biomarker tracking wearable band includes ECG sensors (electrodes) for measuring the ECG signal and a PPG sensor for measuring the optical signal for detecting blood volume changes in arteries and capillaries. The PTT from which systolic and diastolic blood pressure can be derived is the distance between the R peak from the ECG waveform and the systolic phase peak from the PPG signal. That is, the PTT can be derived from two disparate sensors integrated within the single wearable band.

In an implementation, the electrodes in the biomarker tracking wearable band are 10 μm-thick screen-printed Ag-AgCl electrodes on the back (12×12 mm²) and side (6×12 mm²) of the assembled printed circuit board. In an implementation, the back electrode contacts the wrist or like surface and the side electrode is intended to be touched by an index finger of the opposing hand or like for completing the circuit and enabling a single-lead ECG readout.

In an implementation, the biomarker tracking wearable band includes a wireless receive coil for inductive charging of a 3.7V Lithium Ion battery with 20 mAh capacity. A three-light display LED on board provides notifications as to the function of the device (power on, pairing, sensor measurements, low power mode etc.).

The biomarker tracking wearable band incorporates direct over molding of the on-board electronics and assembled printed circuit boards using low temperature silicones with specialized high accuracy mixing equipment. In an implementation, the electronics (printed circuit board (PCB)) comprising active and passive electrical components, the battery and sensors including the ECG and PPG boards are over molded in low temperature liquid silicone rubber. In an implementation, the over molding method includes a 3D printed thermally protective/insulating shroud for direct silicone over molding onto electronics/PCB assemblies (PCBAs), a 3D printed thermally protective/insulating injection plate for direct silicone over molding onto electronics/PCBAs, designed overflow detail to allow for lower fill pressures and provide thorough evacuation of entrapped air (molded overflow detail is removed after molding), and mold designed with adjustable pre-load features for optimization and adjustment of steel seal-off areas on sensors as needed. Silicone is inert and by employing it for over molding of the electronics, the biomarker tracking wearable band can be used in the clinical realm for short term monitoring of patients in an ambulatory setting (e.g. hemodialysis, chemotherapy treatments etc.) where metrics such as HR, HRV and BP require serial monitoring.

The biomarker tracking wearable band has applicability to consumer health, diagnostics, patient monitoring, trauma, carbon monoxide, diagnostics consumables and blood management and can be used for hydration, infection, thermoregulation, muscle fatigue, dialysis, firefighting, hyperhidrosis, stress, and like health, wellness, illness, and disease states.

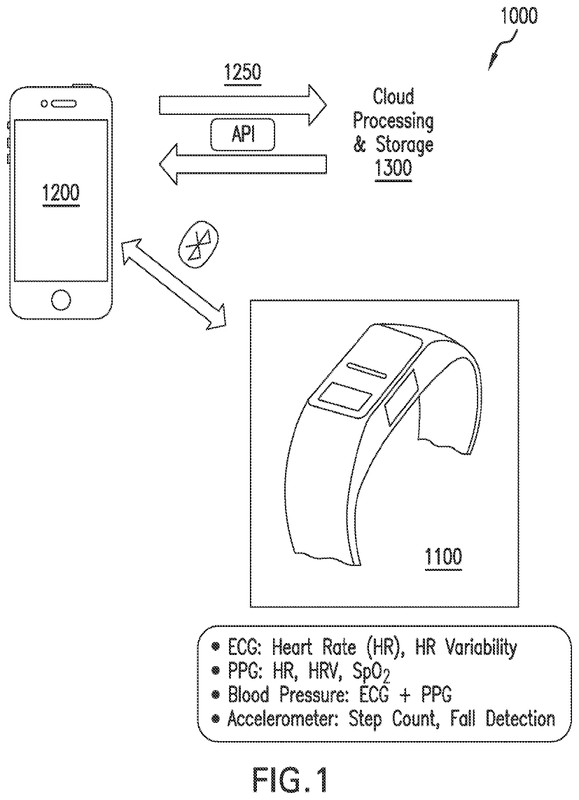

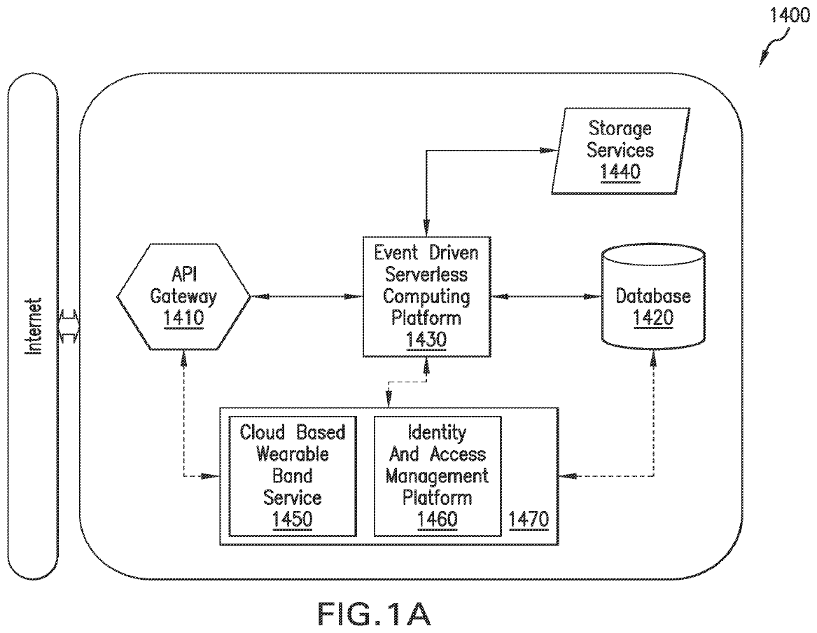

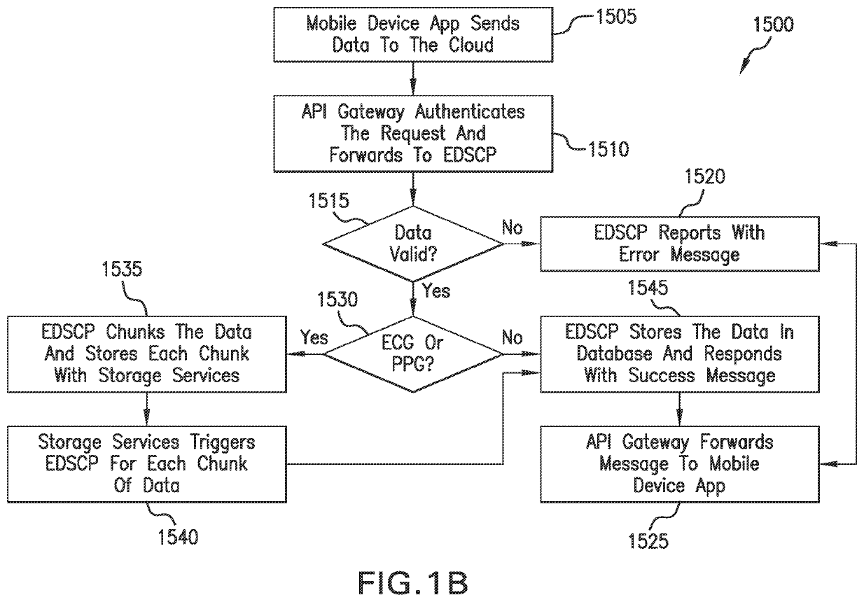

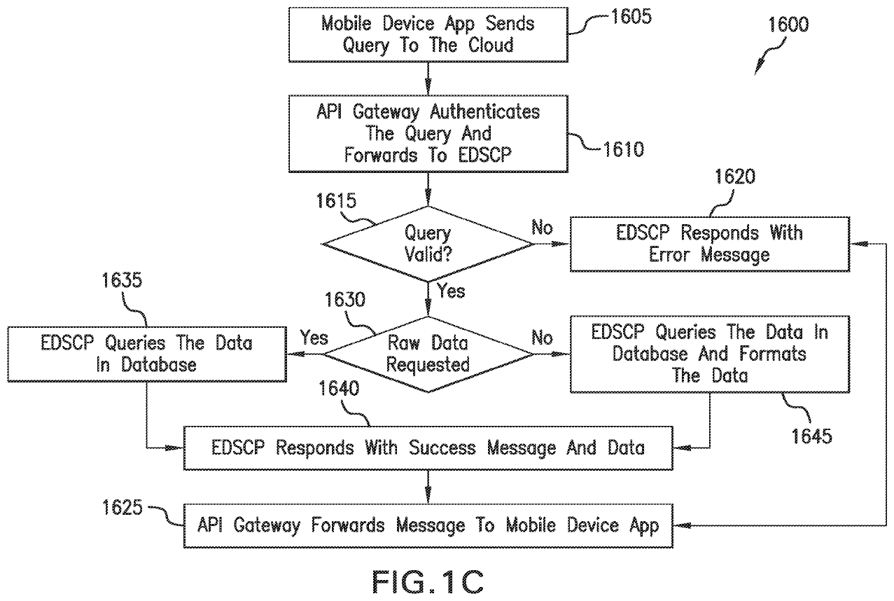

FIG. 1 is an example diagram of an architecture or system 1000 with a biomarker tracking wearable band 1100 in accordance with certain implementations. The system 1000 includes a biomarker tracking wearable band 1100 in communication with or connected to (collectively “connected to”) an application device 1200, which in turn in connected to cloud-based processing and storage devices (collectively “cloud device”) 1300. The biomarker tracking wearable band 1100, the application device 1200, and the cloud device 1300 may be connected via wired, wireless, and/or combinations thereof, where the connections can include networks such as, but not limited to, the Internet, an intranet, a local area network (LAN), a wide area network (WAN), a public network, a private or personal area network such as Bluetooth, a cellular network, a WiFi-based network, a telephone network, a landline network, public switched telephone network (PSTN), a wireless network, a wired network, a private branch exchange (PBX), an Integrated Services Digital Network (ISDN), a IP Multimedia Services (IMS) network, a Voice over Internet Protocol (VoIP) network, and like including any combinations thereof.

The biomarker tracking wearable band 1110 includes a plurality of sensors for measuring physiological, biological, and like measurements of a user. The sensors include, but is not limited to, ECG sensors for measuring heart rate and heart rate variability, PPG sensors for measuring heart rate, heart rate variability, blood pressure and blood oxygen saturation (SpO₂), accelerometers for measuring step count and fall detection, and other like sensors.

The application device 1200 can be, but is not limited to, end user devices, personal computers (PCs), cellular telephones, Internet Protocol (IP) devices, computers, desktop computers, laptops, mobile devices, handheld computers, PDAs, personal media devices, smartphones, notebooks, notepads, phablets and the like which include at least a display and processors for processing and displaying data.

The cloud device 1300 is any cloud computing platform or device which provides processing, storage, and like services. In an implementation, the cloud device 1300 allows the application device 1200 to store and retrieve health/medical data via an API 1250. FIG. 1A is an example diagram of a cloud architecture 1400 for the cloud device 1300 in accordance with certain implementations. The cloud device architecture 1400 includes an API Gateway 1410, a database 1420, an event-driven, serverless computing platform (EDSCP) 1430, storage services 1440, a cloud wearable band service platform 1450, and an identity and access management platform 1460. The API Gateway 1410 is connected to the EDSCP 1430, the cloud wearable band service platform 1450, and the identity and access management platform 1460. The EDSCP 1430 is connected to the cloud wearable band service platform 1450, the storage services 1440, the identity and access management platform 1460, and the database 1420, which in turn is connected to the block 1470 comprising the cloud wearable band service platform 1450 and the identity and access management platform 1460.

The API Gateway 1410 hosts the APIs for the application device 1200 to consume. The database 1420 consists of a NoSQL database with multiple tables to store the sensor data and other information. The EDSCP 1430 runs code in response to events and automatically manages the computing resources required by that code and runs code without provisioning or managing servers.

The cloud device 1300 will store and associate healthcare data with a profile ID. If a user on a mobile application or application device 1200 changes his/her profile ID, old data is associated with old profile ID and new data will be associated with the new profile ID. There is no aggregation of profile IDs. All API's use JSON format. The mobile application or application device 1200 will send data to the cloud device 1300 for a specific day, or portion thereof, at a time. In other words, a single API request to store data will only include data for a specific day but may send multiple API requests for the same day. The mobile application or application device 120 will send data to the cloud device 1300 as soon as possible (when connected to the network). The cloud API's are asynchronous and will store data as they are received and will allow the retrieval of data as they are requested.

The API 1250 is used to store user profile information. The mobile application or application device 1200 allows a user to enter the following information during initial registration: Gender, Age, Height, Weight, and a unique username (e.g. a user-entered email address). The mobile application or application device 1200 will convert this username to a unique ID using a pre-defined algorithm. The user is not allowed to change the username. The API 1250 will use only the ID. This mechanism will allow a user to view his/her information on a web app, when available, where the web app can use the same pre-defined algorithm to convert the user-provided username to a profile ID that can be used in the API request. When the user submits the information, the mobile application or application device 1200 stores them locally and sends them to the cloud device 1300. The mobile application or application device 1200 will send the ID and other data such as gender, height, and weight, when changed. When the user updates this information, the mobile application or application device 1200 will send the information to the cloud device 1300.

FIG. 1B is an example flowchart of a method 1500 for cloud storage in a cloud architecture for a biomarker tracking wearable band in accordance with certain implementations. A mobile device, for example, sends data, for example, biomarker tracking data, to the cloud (1505). The API gateway authenticates the request and forwards the data to the EDSCP (1510). The EDSCP determines the validity of the data (1515). If the data is invalid, the EDSCP sends an error message to the API gateway (1520), which in turn forwards a message to the mobile device (1525). If the data is valid, the EDSCP determines what type of data (1530). If the data is ECG data or PPG data, the EDSCP segments the data and stores each segment in the storage services (1535). The storage services triggers the EDSCP to store each segment of data in the database (1540). The EDSCP sends a success message after each successful storage event (1545). If the data is not ECG data or PPG data, the EDSCP stores the data in the database and sends a success message after each successful storage event (1545). The API gateway forwards the messages to the mobile device (1525).

FIG. 1C is an example flowchart of a method 1600 for querying cloud storage in a cloud architecture for a biomarker tracking wearable band in accordance with certain implementations. A mobile device queries the cloud for data (1605). The API gateway authenticates the query and forwards the query to the event-driven, serverless computing platform (1610). If the query is not valid, the EDSCP sends an error message to the API gateway (1615), which in turn forwards the error message to the mobile device (1620). If the query is valid, the EDSCP determines if the request is for raw data (1625). If the request is for raw data, then the EDSCP queries the database (1635). The EDSCP then responds with the received data and a success message (1640), which is forwarded to the API gateway and then to the mobile device (1625). If the request is not for raw data, then the EDSCP queries the database and formats the received data (1645). The EDSCP then responds with the formatted data and a success message (1640), which is forwarded to the API gateway and then to the mobile device (1625).

Operationally with respect to FIGS. 1, 1A, 1B, and 1C, a user is provided with the biomarker tracking wearable band. The biomarker tracking wearable band collects data for and from the user using the on-board sensors. The data is transmitted, sent, and/or communicated to the application device. In an implementation, the data is transmitted via a BlueTooth® connection with the application device. The application device can process the data to determine physiological and biological characteristics of the user such as blood pressure, which is determined from a pulse transit time (PTT) derived from simultaneous ECG and PPG measurements. In an implementation, other physiological and biological characteristics can be determined. The application device can display the measured and derived physiological and biological characteristics or parameters of the user. The measured and derived physiological and biological characteristics or parameters can be transmitted, sent, and/or communicated by the application device to the cloud device for analysis and storage. For example, the cloud device can perform historical analysis on the measured and derived physiological and biological characteristics or parameters and provide recommendations to the user via the application device.

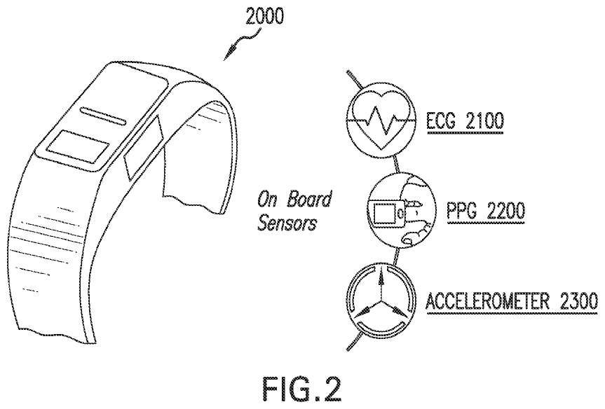

FIG. 2 is an example diagram of a biomarker tracking wearable band 2000 in accordance with certain implementations. As described herein, the biomarker tracking wearable band 2000 includes an ECG sensor 2100, a PPG sensor 2200, and accelerometer 2300. In this instance, the biomarker tracking wearable band 2000 is in a force-form fit form factor. Operationally, the biomarker tracking wearable band 2000 can function as described herein.

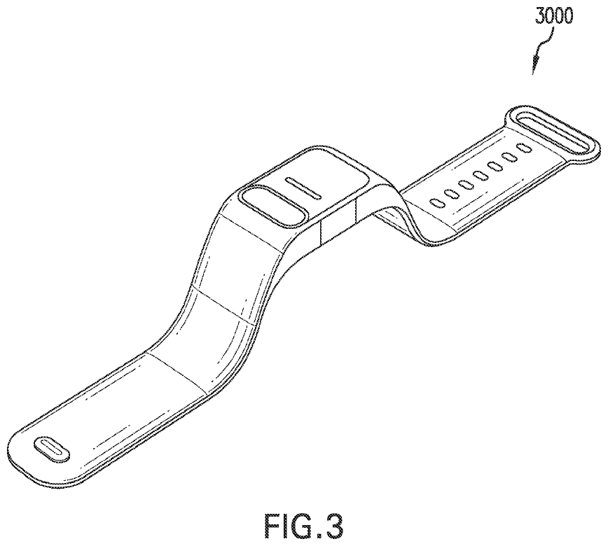

FIG. 3 is an example diagram of a biomarker tracking wearable band 3000 in accordance with certain implementations. In this instance, the biomarker tracking wearable band 3000 is in a belted form factor. Operationally, the biomarker tracking wearable band 3000 can include the components described herein and function as described herein.

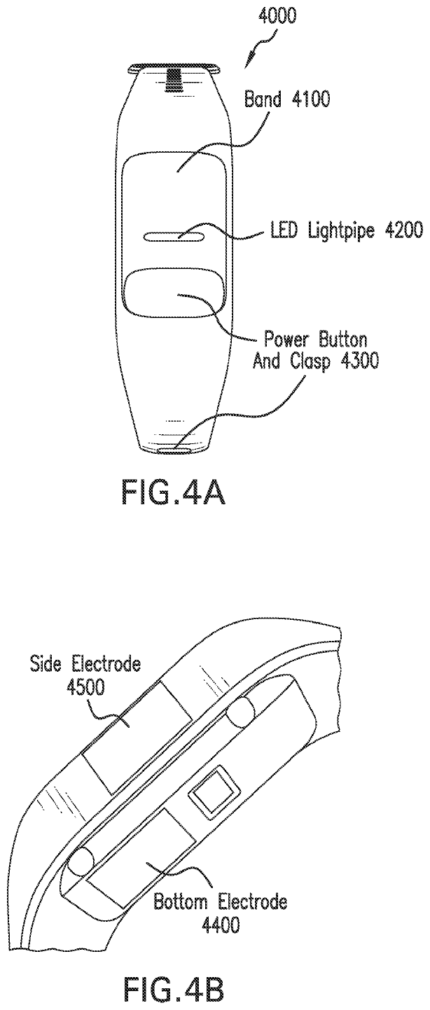

FIGS. 4A-B are example diagrams of a biomarker tracking wearable band 4000 in accordance with certain implementations. As shown in FIG. 4A, the biomarker tracking wearable band 4000 includes a band for engagement with a user 4100, a LED lightpipe or display 4200 for indicating different actions, and a power button 4300. As shown in FIG. 4B, the biomarker tracking wearable band 4000 includes a bottom or user surface facing electrode 4400 and a side electrode 4500 for an ECG sensor.

In an implementation, the LED display 4200 includes three LEDs, each having a different color. The three LEDs can include a green LED, a red LED, and a blue LED. Consequently, the LED display 4200 can indicate a number of actions depending on the color or sequencing of the LEDs. In an implementation, if the LED display 4200 is green, this indicates that the biomarker tracking wearable band 4000 is powered on. The LED display 4200 remains ‘ON’ or green for 2 seconds and thereafter blinks once every 2 seconds to indicate normal operation. In an implementation, if the LED display 4200 is green-blue, this indicates that the biomarker tracking wearable band 4000 is in the process of Bluetooth® pairing with an application device, for example. In this instance, there is one blue blink added to normal green blinks (as above) at the point of pairing. After pairing, the LED display 4200 reverts back to blinking green only with one blink every 2 seconds. If the pairing is unsuccessful, the LED display 4200 reverts back to blinking green only with one blink every 2 seconds. In an implementation, if the LED display 4200 is red, this indicates that the biomarker tracking wearable band 4000 is charging. The biomarker tracking wearable band 4000 may be required to remain ‘ON’ for enabling this use case. A steady light (remains ‘ON’) for the duration of the charging cycle (e.g. when device is placed on charge pad).

In an implementation, if the LED display 4200 is blue, this indicates that a fall detection event has occurred. The LED display 4200 remains ‘ON’ for 2 seconds. After the fall detection, the LED display 4200 reverts back to blinking green once every 2 seconds for normal operation. In an implementation, if the LED display 4200 is red, this indicates that the power of the biomarker tracking wearable band is low. At low power levels, the LED display 4200 blinks twice separated by 500 milliseconds between each blink (for a total duration of 1 second). The frequency of the 2 blinks is once every 10 seconds.

In an implementation, if the LED display 4200 is blue, this indicates that only a PPG sensor is acquiring data. In this instance, the LED display 4200 blinks once every 250 milliseconds during PPG only acquisition (4 blinks in 1 second). Blinking at the above rate continues until acquisition is terminated or heart rate and heart rate variability values are provided on the application device. In an implementation, PPG LEDs may also be lighting up on the backside of the biomarker tracking wearable band 4000 during this measurement.

In an implementation, if the LED display 4200 is blue, this indicates that an ECG sensor is acquiring data. In this instance, the LED display 4200 blinks once every 500 milliseconds during ECG only acquisition (2 blinks in 1 second). The blinking at the above rate continues until acquisition is terminated or the heart rate and heart rate variability values provided to the application device, for example. In this instance, users will have to position index finger from opposing hand on side electrode to complete this measurement. In an implementation, if the LED display 4200 is blue, this indicates that simultaneous or nearly simultaneous measurements are being made to derive blood pressure (BP) based on PTT. In this instance, the LED display 4200 remains ‘ON’ (steady light) for the entirety of the measurement until systolic and diastolic BP values are displayed on the application device or until the measurements are terminated. In this instance, users will have to maintain finger position on side electrode and may observe PPG LEDs on the backside of the device. In an implementation, if the LED display 4200 is dark or has no visible light, this indicates that the biomarker tracking wearable band is powered off. There is no fixed length of time required prior to switching the device OFF. Operationally, the biomarker tracking wearable band 4000 can include the components described herein and function as described herein.

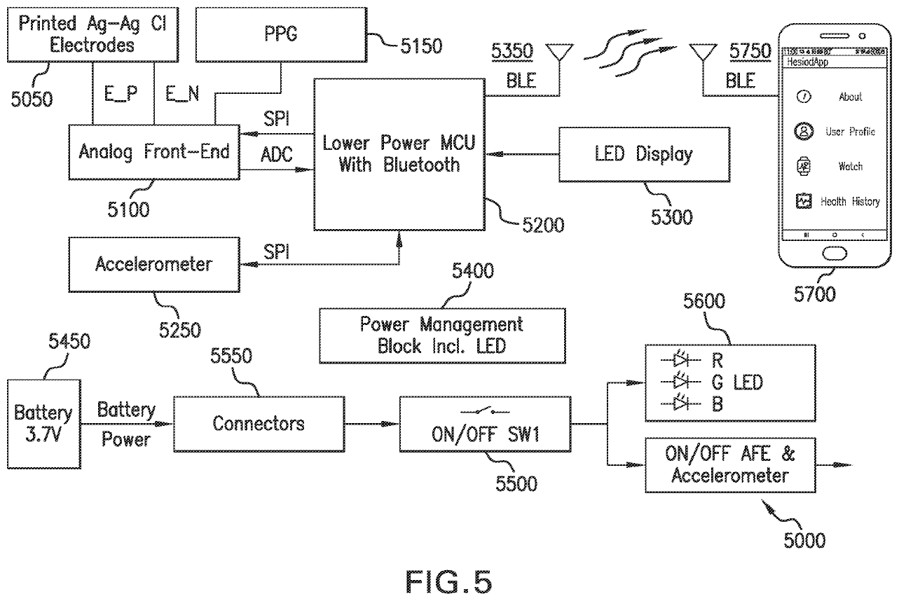

FIG. 5 is an example diagram of a hardware architecture of a biomarker tracking wearable band 5000 in accordance with certain implementations. The biomarker tracking wearable band 5000 includes printed silver (Ag)-silver chloride (AgCl) electrodes 5050 (for the ECG measurements) which are connected to an analog front-end 5100. A PPG sensor 5150 is also connected to the analog front-end 5100. The analog front-end 5100 is connected to a processor 5200 (a low power MCU with integrated Bluetooth®), which is further connected to an accelerometer 5250. a LED display 5300 and to an antenna 5350. The biomarker tracking wearable band 5000 also includes a power management block 5400. The biomarker tracking wearable band 5000 further includes a battery 5450 which is connected to an on/off switch 5500 via connectors 5550. The on/off switch 5500 is further connected to red, blue, and green LEDS 5600, and to the analog front-end 5250 and the accelerometer 5100. Operationally, the biomarker tracking wearable band 5000 can include the components described herein and function as described herein. The biomarker tracking wearable band 5000 can communicate via the antenna 5350 with a device 5700 which has an antenna 5750. Operationally, the biomarker tracking wearable band 5000 can include the components described herein and function as described herein.

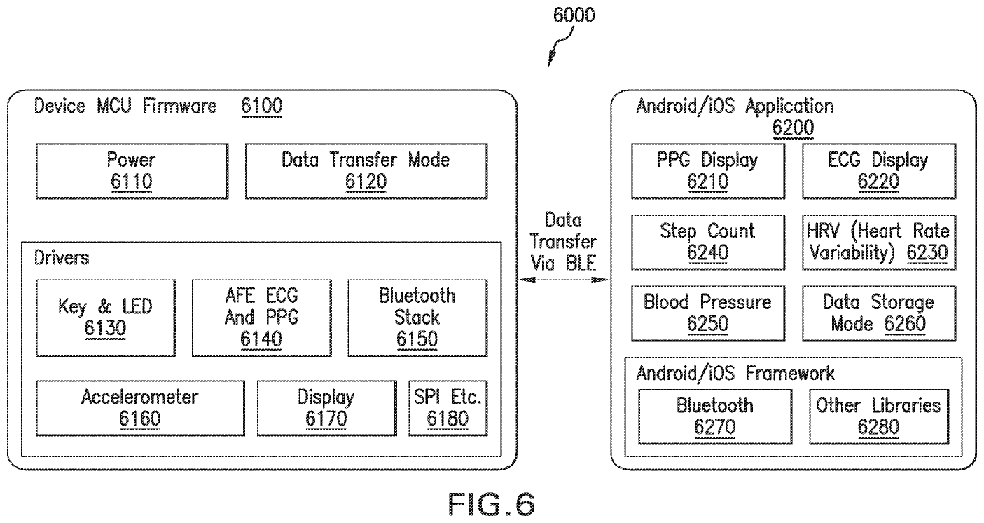

FIG. 6 is an example diagram of a software and firmware architecture 6000 of a biomarker tracking wearable band and application device in accordance with implementations. A processor firmware 6100 of the biomarker tracking wearable band includes power 6110 and data transfer 6120 modules and drivers for the LEDs 6130, analog front-end, ECG sensor, and PPG sensors 6140, Bluetooth® stack 6150, accelerometer 6160, display 6170, serial peripheral interface 6180, and the like. The application device 6200 includes applications to process and display PPG data 6210, ECG data 6220, heart rate variability data 6230, step count 6240, blood pressure 6250 and like data. The application device 6200 further includes a data storage mode 6260, a Bluetooth® stack 6270, and other libraries 6280. Operationally, the biomarker tracking wearable band including the software and firmware architecture 6000 can include the components described herein and function as described herein.

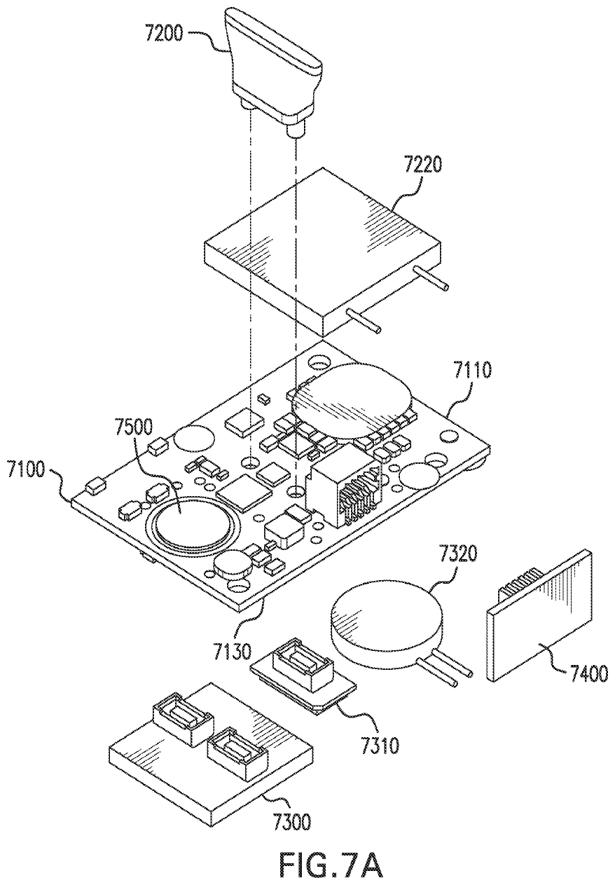

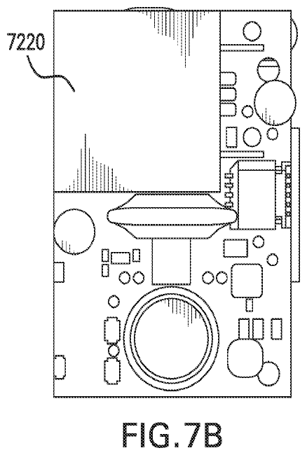

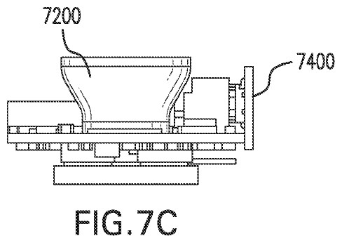

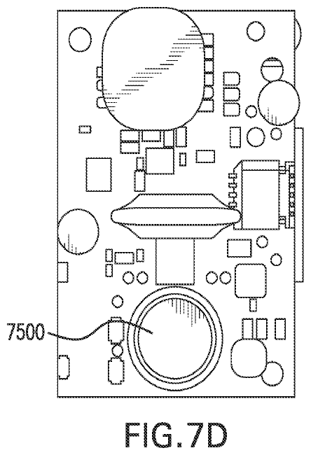

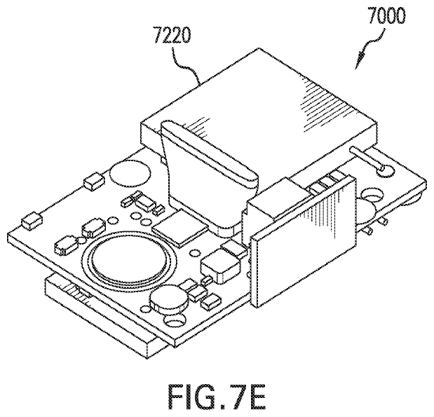

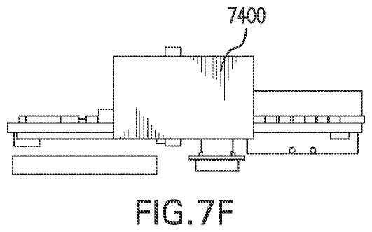

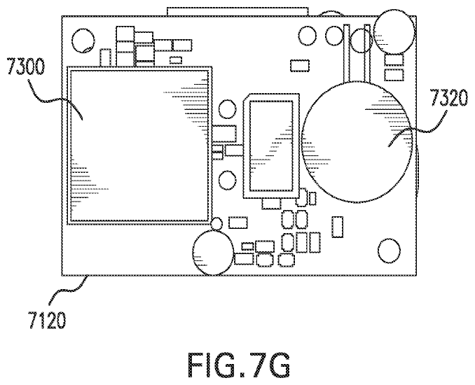

FIG. 7 is an example diagram or layout of a printed circuit board assembly (PCBA) 7100 of a biomarker tracking wearable band 7000 in accordance with implementations which includes FIGS. 7A, 7B, 7C, 7D, 7E, 7F, and 7G. In an exploded view, the biomarker tracking wearable band 7000 includes a lightpipe 7200 and a battery 7210 which populate a top surface 7110 of the main board of the PCBA 7100. In the exploded view, the biomarker tracking wearable band 7000 includes a bottom ECG electrode 7300, a PPG sensor 7310, and a wireless power charger 7320 which populate a bottom surface 7120 of the main board of the PCBA 7100. In the exploded view, the biomarker tracking wearable band 7000 includes a side ECG electrode 7400 which is connected to the side 7130 of the main board of the PCBA 7100. The top surface 7110 of the PCBA 7100 also includes a power button 7500. Operationally, the biomarker tracking wearable band including the software and firmware architecture 7000 can include the components described herein and function as described herein.

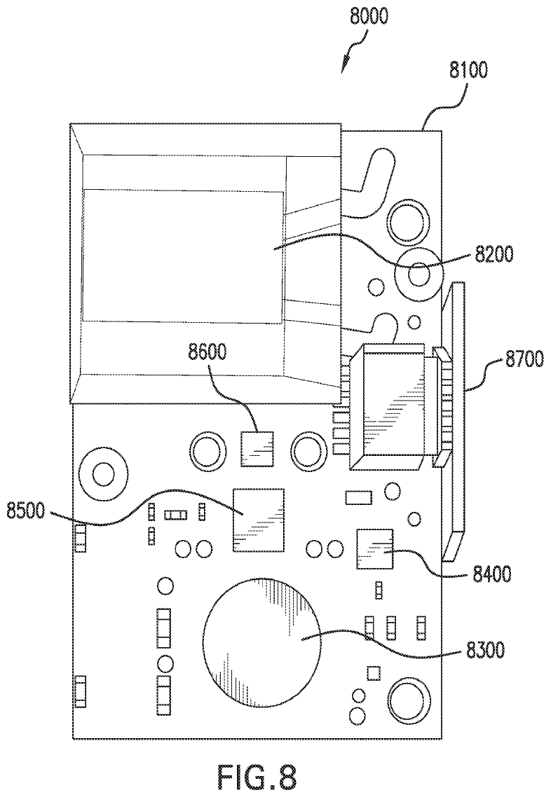

FIG. 8 is an example diagram or layout of a top view of a PCBA 8100 of a biomarker tracking wearable band 8000 in accordance with implementations. This view shows a battery pack 8200, a power switch 8300, an accelerometer 8400, a processor 8500, a display LED 8600, and a screen printed side ECG Ag-AgCl electrode 8700. Operationally, the biomarker tracking wearable band 8000 can include the components described herein and function as described herein.

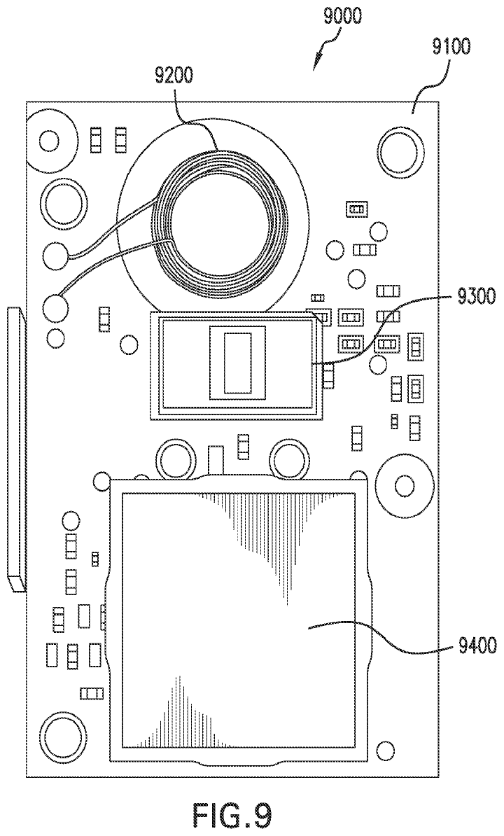

FIG. 9 is an example diagram or layout of a bottom view of a PCBA 9100 of a biomarker tracking wearable band 9000 in accordance with implementations. This view shows a wireless receiver coil 9200 for inductive charging, a PPG sensor 9300 for optical measurements of the heart rate and oxygen saturation, and the screen printed bottom or back ECG Ag-AgCl electrode 9400 for contact with a wrist, for example. Operationally, the biomarker tracking wearable band 9000 can include the components described herein and function as described herein.

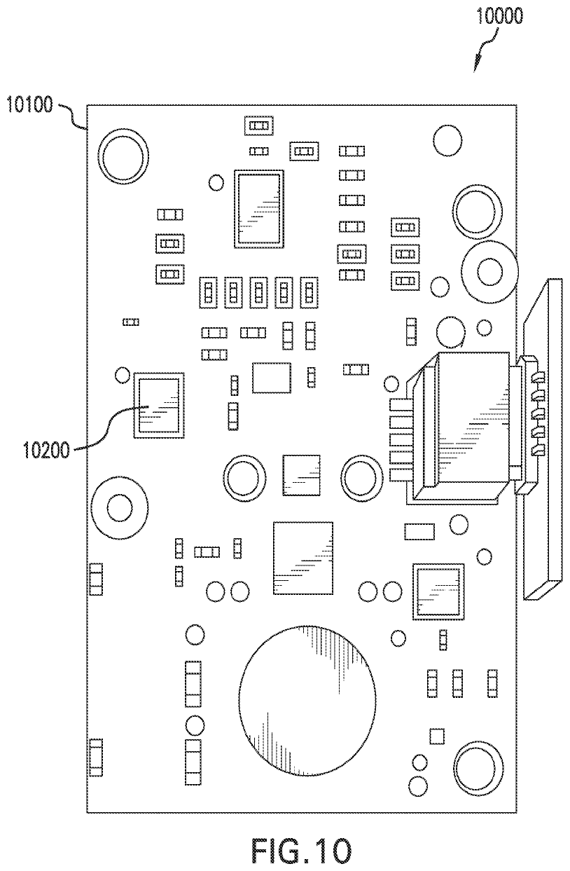

FIG. 10 is an example diagram or layout of a top view of a PCBA 10100 without a battery of a biomarker tracking wearable band 10000 in accordance with implementations. In this view, a battery pack is removed, and an analog front end 10200 that is used for PPG and ECG signal processing is shown. Operationally, the biomarker tracking wearable band 10000 can include the components described herein and function as described herein.

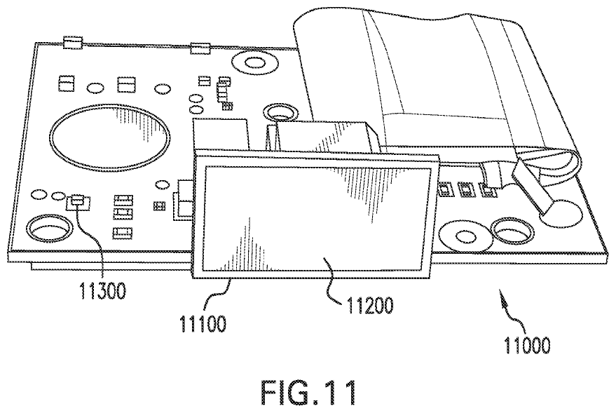

FIG. 11 is an example diagram or layout of a side view of a PCBA 11100 of a biomarker tracking wearable band 11000 in accordance with implementations. In this view, a screen-printed side ECG Ag-AgCl electrode 11200 for contact with index finger of opposing hand is shown, along with a low dropout voltage regulator 11300. Operationally, the biomarker tracking wearable band 11000 can include the components described herein and function as described herein.

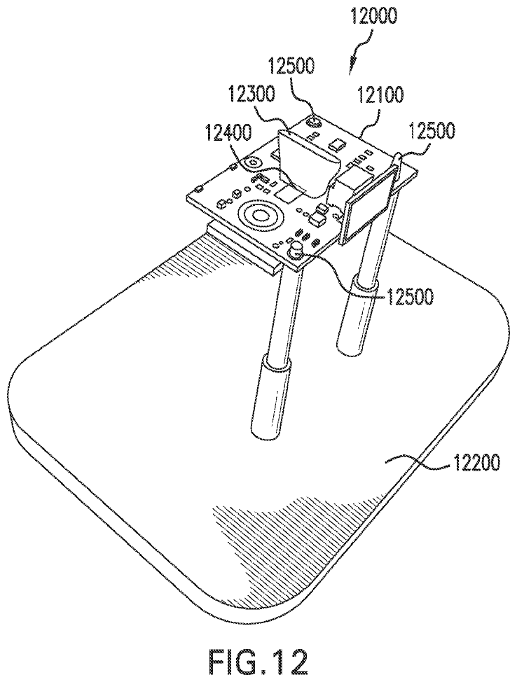

FIG. 12 is an example photograph of a perspective view of a PCBA 12100 in a holder 12200 of a biomarker tracking wearable band 12000 in accordance with implementations. In this view, a lightpipe 12300 is positioned over a display LED 12400. In addition, the PCBA 12100 includes three holes 12500 for engaging a mold as described herein. In implementations, the lightpipe 12400 is 3D printed. Operationally, the biomarker tracking wearable band 12000 can include the components described herein and function as described herein.

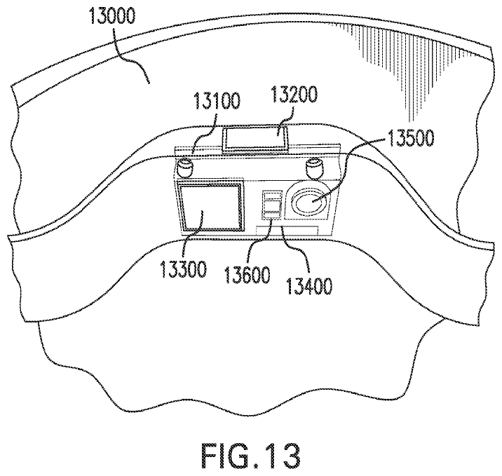

FIG. 13 is an example photograph of a biomarker tracking wearable band 13000 in accordance with implementations. In this instance, a PCBA 13100 has been directly over molded using a clear low temperature mixture as described herein. Consequently, a screen printed side ECG Ag-AgCl electrode 13200, a screen printed bottom ECG Ag-AgCl electrode 13300, a PPG sensor 13400, a wireless receive charging coil 13500 are visible. In this instance, the LED—photodiode bank 13600 is also visible. As implemented, the PPG sensor 13400 employs reflectance pulse oximetry to make the appropriate measurements. Operationally, the biomarker tracking wearable band 13000 can include the components described herein and function as described herein.

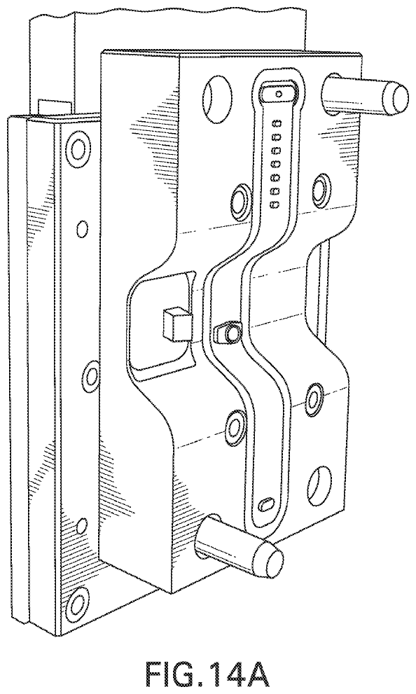

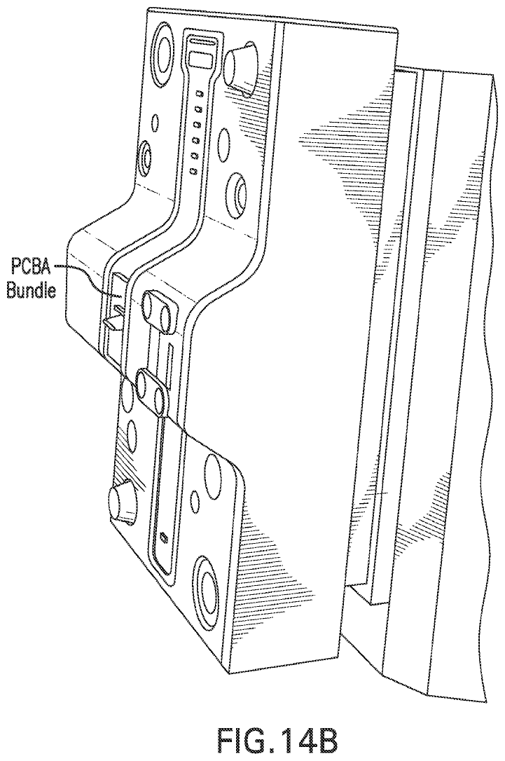

FIGS. 14A and 14B are photographs of a mold for use in over molding for a biomarker tracking wearable band in accordance with certain implementations. FIG. 14A shows the cavity side of the mold and FIG. 14B shows the core side of the mold. In this instance, a PCBA is held in place on the mold using the three holes in the PCBA. The mold is used along with a low temperature curing silicone having a durometer of 70 Shore A. This enables direct over molding onto the PCBA. The silicone is mixed with additives including a catalyst, control, accelerant (if used), and color. These materials are mixed on top of a mold machine, at the machine's injection screw/barrel. When the catalyst in the silicone and the control mix together, the silicone begins to cure. The overall speed of the curing process is regulated by the ratio of control to accelerant.

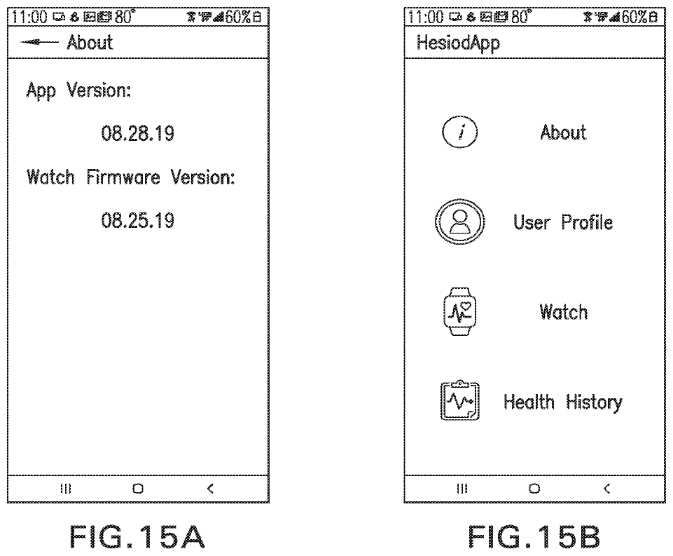

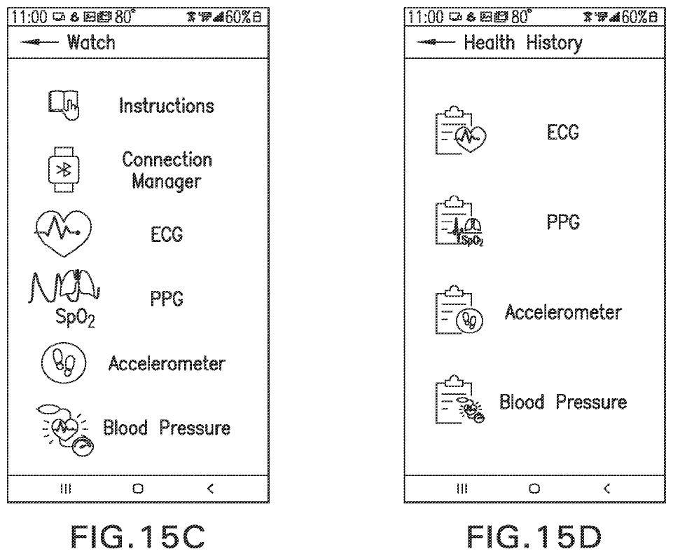

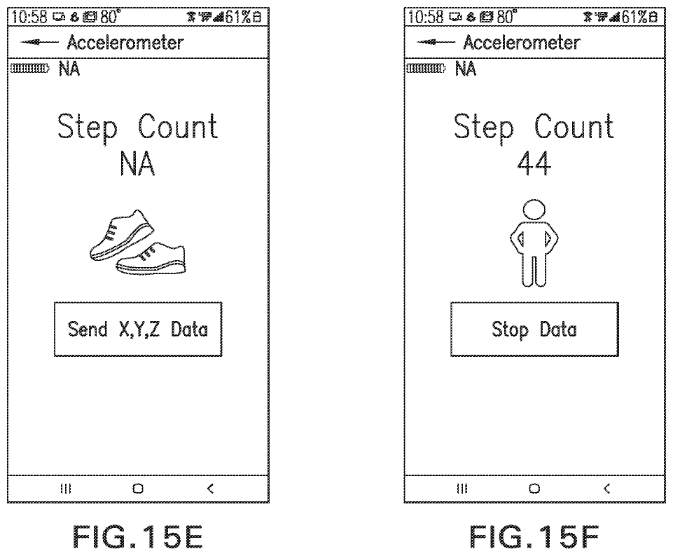

FIGS. 15A-F are example diagrams of interface screens on a device for interacting with a biomarker tracking wearable band in accordance with implementations. FIG. 15A is a screenshot of the version page, FIG. 15B is a screenshot of the main page, FIG. 15C is a screenshot after clicking on a wearable band link, FIG. 15D is a screenshot after clicking on a history link, FIG. 15E is a screenshot after clicking on an accelerometer link, and FIG. 15F is a screenshot after clicking on the accelerometer link and viewing the actual step count.

In an illustrative clinical (equilibrium state) use case, an accelerometer is turned off and PPG and ECG sensors are not active. If the user presses the start button for ECG sensing and the circuit is complete, ECG sensor starts measuring the electrical activity of the heart. This invokes the PPG sensor to detect the volume of blood flow. The accelerometer is activated to record any present data. The data is sent to an application/application device using Bluetooth Interface (BLE) to observe the data in real time. Application records & shows data in real time.

In an illustrative active state use case, a use case is entered when the user has been involved in a fitness activity and starts the device. The user could start the device when in motion or following the activity. ECG cannot be performed when the user is in motion. In this case, the accelerometer is turned off and the PPG and ECG sensors are not active. If the user presses the start button for ECG sensing and the user is in motion, then the circuit is not completed, and ECG cannot be started. The pressing of the start button invokes the PPG sensor to detect the volume of blood flow. The system waits for the circuit to complete to start ECG. The accelerometer is activated to record any present data sensing. The data is sent to an application/application device using Bluetooth Interface (BLE) to observe the data in real time. Application records & shows data in real time.

In an illustrative inactive state use case, the accelerometer and PPG is active and ECG sensing is not active. The device operates in low power mode to record the data in this mode. PPG and accelerometer continue to stay active to monitor sleep wake patterns. The data is sent to an application/application device using Bluetooth Interface (BLE) to record the data. Application shows data in real time for accelerometer and PPG.

The construction and arrangement of the methods as shown in the various exemplary embodiments are illustrative only. Although only a few embodiments have been described in detail in this disclosure, many modifications are possible (e.g., variations in sizes, dimensions, structures, shapes and proportions of the various elements, values of parameters, mounting arrangements, use of materials and components, colors, orientations, etc.). For example, the position of elements may be reversed or otherwise varied and the nature or number of discrete elements or positions may be altered or varied. Accordingly, all such modifications are intended to be included within the scope of the present disclosure. The order or sequence of any process or method steps may be varied or re-sequenced according to alternative embodiments. Other substitutions, modifications, changes, and omissions may be made in the design, operating conditions and arrangement of the exemplary embodiments without departing from the scope of the present disclosure.

Although the figures may show a specific order of method steps, the order of the steps may differ from what is depicted. Also two or more steps may be performed concurrently or with partial concurrence. Such variation will depend on the software and hardware systems chosen and on designer choice. All such variations are within the scope of the disclosure. Likewise, software implementations could be accomplished with standard programming techniques with rule-based logic and other logic to accomplish the various connection steps, processing steps, comparison steps, and decision steps.

While the disclosure has been described in connection with certain embodiments, it is to be understood that the disclosure is not to be limited to the disclosed embodiments but, on the contrary, is intended to cover various modifications and equivalent arrangements included within the scope of the appended claims, which scope is to be accorded the broadest interpretation so as to encompass all such modifications and equivalent structures as is permitted under the law.

1. A biomarker tracking wearable band comprising:
a printed circuit board assembly (PCBA), the PCBA including:
an electrocardiography (ECG) sensor utilizing printed Silver-Silver Chloride (Ag-AgCl)
electrodes; and
an optical photoplethysmography (PPG) sensor utilizing more than two light
emitting diodes (LEDs); and
a directly over molded band encasing the PCBA.

2. The biomarker tracking wearable band of claim 1, wherein the printed Silver-Silver Chloride (Ag-AgCl) electrodes further comprising:
a side Ag-AgCl electrode configured to be contacted by a finger; and
a bottom Ag-AgCl electrode configured for contact with an appendage,
wherein placement of the finger on the side Ag-AgCl electrode completes a circuit for enabling a single-lead ECG readout.

3. The biomarker tracking wearable band of claim 2, further comprising:
an accelerometer configured to monitor user activity including at least step counts and body posture; and
a lightpipe configured over the more than two LEDs.

4. The biomarker tracking wearable band of claim 3, wherein the more than two light LEDs and one or more photodiodes are on a same plane to implement reflectance oximetry and the one or more photodiodes measure backscatter of light.

5. The biomarker tracking wearable band of claim 4, wherein the directly over molded band comprises low temperature silicones mixed with additives including at least a catalyst, a control, an accelerant, and color using a mold machine, wherein the silicone begins to cure when the catalyst in the silicone and the control mix together.

6. The biomarker tracking wearable band of claim 5, wherein the PCBA comprises a plurality of holes for alignment in the mold machine.

7. A system for tracking biomarkers, comprising:
a biomarker tracking wearable band comprising:
a printed circuit board assembly (PCBA), the PCBA including:
an electrocardiography (ECG) sensor utilizing printed Silver-Silver Chloride (Ag-AgCl) electrodes; and
an optical photoplethysmography (PPG) sensor utilizing more than two
light emitting diodes (LEDs); and
a directly over molded band encasing the PCBA; and
a device configured to receive data from the biomarker tracking wearable band, the device configured to:
present heart rate (HR) and heart rate variability (HRV) from the ECG sensor;
present HR, HRV and SpO₂ measurements from the PPG sensor; and
present blood pressure measurements on the basis of a pulse transit time (PTT) derived from ECG sensor waveform data decomposition and PPG sensor waveform data decomposition.

8. The system of claim 7, wherein the printed Silver-Silver Chloride (Ag-AgCl) electrodes further comprising:
a side Ag-AgCl electrode configured to be contacted by a finger; and
a bottom Ag-AgCl electrode configured for contact with an appendage,
wherein placement of the finger on the side Ag-AgCl electrode completes a circuit for enabling a single-lead ECG readout.

9. The system of claim 8, wherein the biomarker tracking wearable band further comprising an accelerometer configured to monitor user activity including at least step counts and body posture and a lightpipe configured over the more than two LEDs, the device configured to present step counts and body posture from the accelerometer.

10. The system of claim 9, wherein the more than two light LEDs and one or more photodiodes are on a same plane to implement reflectance oximetry and the one or more photodiodes measure backscatter of light.

11. The system of claim 10, wherein the directly over molded band comprises low temperature silicones mixed with additives including at least a catalyst, a control, an accelerant, and color using a mold machine, wherein the silicone begins to cure when the catalyst in the silicone and the control mix together.

12. The system of claim 11, wherein the PCBA comprises a plurality of holes for alignment in the mold machine.

13. The system of claim 12, further comprising:
a cloud system configured to receive and transmit data with the device, wherein the data includes at least historical sensor data and biomarker data.

14. A method for making tracking biomarkers, the method comprising:
printing Silver-Silver Chloride (Ag-AgCl) electrodes on a printed circuit board (PCB) to provide electrocardiography (ECG) measurements;
provisioning an optical photoplethysmography (PPG) sensor on the PCB to provide optical measurements of the heart rate and oxygen saturation, wherein the PPG uses more than two light emitting diodes (LEDs); and
directly over molding a band encasing the PCB with the Ag-AgCl electrodes and the PPG sensor.

15. The method of claim 14, wherein the directly over molding further comprises:
providing a 3D printed thermally protective and insulating shroud for direct silicone over molding onto the PCB; and
providing a 3D printed thermally protective and insulating injection plate for direct silicone over molding onto the PCB.

16. The method of claim 15, wherein the printing further comprises:
printing a side Ag-AgCl electrode configured to be contacted by a finger; and
printing a bottom Ag-AgCl electrode configured for contact with an appendage, wherein placement of the finger on the side Ag-AgCl electrode completes a circuit for enabling a single-lead ECG readout.

17. The method of claim 16, wherein the band comprises low temperature silicones mixed with additives including at least a catalyst, a control, an accelerant, and color using a mold machine, wherein the silicone begins to cure when the catalyst in the silicone and the control mix together.

18. The method of claim 17, further comprising:
providing an accelerometer on the PCB to monitor user activity including at least step counts and body posture; and
providing a lightpipe configured over the more than two LEDs.

19. The method of claim 18, further comprising:
providing one or more photodiodes to measure backscatter of light, wherein the more than two light LEDs and are on a same plane to implement reflectance oximetry.

20. The method of claim 18, further comprising:
providing the PCB with a plurality of holes for alignment in the mold machine.
